(19) United States
(12) Patent Application Publication
(10) Pub. No.: US 2006/0258640 A1
(43) Pub. Date: Nov. 16, 2006

(54) Use of Flibanserin in the treatment of chronic pain

(75) Inventors: Angelo CECI, Mittelbiberach (DE); Franco BORSINI, Ardea, Rome (IT); Wolfram GAIDA, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/381,590

(22) Filed: May 4, 2006

(30) Foreign Application Priority Data
May 13, 2005 (EP) 05010437

(51) Int. Cl. A61K 31/55; A61K 31/485; A61K 31/4166; A61K 31/192; A61K 31/195; A61K 31/445

(52) U.S. Cl. 514/217; 514/282; 514/389; 514/317; 514/571; 514/561

(57) ABSTRACT

The invention relates to a method for the treatment of chronic pain comprising the administration of a therapeutically effective amount of flibanserin

The invention relates to a method for the treatment of chronic pain comprising the administration of a therapeutically effective amount of flibanserin. The invention relates further to new pharmaceutical compositions for the treatment of chronic pain and methods for the preparation thereof. In one embodiment, the instant invention is directed to pharmaceutical combinations comprising flibanserin as one active ingredient in combination with at least one additional active ingredient for the treatment of chronic pain and methods for the preparation thereof.

### DESCRIPTION OF THE INVENTION

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

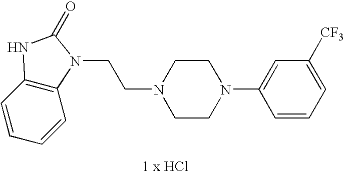

Flibanserin shows affinity for the 5-HT_{1A} and 5-HT₂-receptor. It is therefore a promising therapeutic agent for the treatment of a variety of diseases, for instance depression, schizophrenia, and anxiety.

Surprisingly it has been found that Flibanserin 1, optionally in form of the free base, the pharmacologically acceptable acid addition salts and/or optionally in form of the hydrates and/or solvates thereof proved to be effective in the treatment of chronic pain.

Chronic pain is a pain condition beyond the normal cause of an injury or illness and may be a consequence of inflammation or serious, progressive, painful disease stages. Various types of chronic pain include, but are not limited to, neuropathic pain, diabetic neuropathy, post-herpetic neuralgia (PHN), carpal tunnel syndrome (CTS), HIV neuropathy, phantom limb pain, complex regional pain syndrome (CPRS), trigeminal neuralgia/trigeminus neuralgia/tic douloureux, surgical intervention (e.g. post-operative analgesics), diabetic vasculopathy, capillary resistance or diabetic symptoms associated with insulitis, pain associated with angina, pain associated with menstruation, pain associated with cancer, dental pain, headache, migraine, trigeminal neuralgia, temporomandibular joint syndrome, myofascial pain muscular injury, fibromyalgia syndrome, bone and joint pain (osteoarthritis), rheumatoid arthritis, rheumatoid arthritis and edema resulting from trauma associated with burns, sprains or fracture bone pain due to osteoarthritis, osteoporosis, bone metastases or unknown reasons, gout, fibrositis, myofascial pain, thoracic outlet syndromes, upper back pain or lower back pain (wherein the back pain results from systematic, regional, or primary spine disease (radiculopathy), pelvic pain, cardiac chest pain, non-cardiac chest pain, spinal cord injury (SCI)-associated pain, central post-stroke pain, cancer neuropathy, AIDS pain, sickle cell pain or geriatric pain.

Accordingly, the instant invention relates to a method for the treatment of chronic pain comprising the administration of a therapeutically effective amount of flibanserin 1, optionally in form of the free base, the pharmacologically acceptable acid addition salts and/or optionally in form of the hydrates and/or solvates thereof.

In a preferred embodiment the present invention relates to a method for the treatment of chronic pain, wherein the type of chronic pain is selected from the group consisting of neuropathic pain, diabetic neuropathy, post-herpetic neuralgia (PHN), carpal tunnel syndrome (CTS), HIV neuropathy, phantom limb pain, complex regional pain syndrome (CPRS), trigeminal neuralgia/trigeminus neuralgia/tic douloureux, surgical intervention (e.g. post-operative analgesics), diabetic vasculopathy, capillary resistance or diabetic symptoms associated with insulitis, pain associated with angina, pain associated with menstruation, pain associated with cancer, dental pain, headache, migraine, trigeminal neuralgia, temporomandibular joint syndrome, myofascial pain muscular injury, fibromyalgia syndrome, bone and joint pain (osteoarthritis), rheumatoid arthritis, rheumatoid arthritis and edema resulting from trauma associated with burns, sprains or fracture bone pain due to osteoarthritis, osteoporosis, bone metastases or unknown reasons, gout, fibrositis, myofascial pain, thoracic outlet syndromes, upper back pain or lower back pain (wherein the back pain results from systematic, regional, or primary spine disease (radiculopathy), pelvic pain, cardiac chest pain, non-cardiac chest pain, spinal cord injury (SCI)-associated pain, central post-stroke pain, cancer neuropathy, AIDS pain, sickle cell pain and geriatric pain comprising the administration of a therapeutically effective amount of flibanserin 1, optionally in form of the free base, the pharmacologically acceptable acid addition salts and/or optionally in form of the hydrates and/or solvates thereof.

Especially preferred is a method for the treatment of neuropathic pain comprising the administration of a therapeutically effective amount of flibanserin 1, optionally in form of the free base, the pharmacologically acceptable acid addition salts and/or optionally in form of the hydrates and/or solvates thereof.

Another embodiment of the invention relates to the use of flibanserin 1, optionally in form of the free base, the pharmacologically acceptable acid addition salts and/or optionally in form of the hydrates and/or solvates thereof for the preparation of a medicament for the treatment of any of the aforementioned conditions.

As flibanserin cannot only be used as a monotherapy, but can also be used in combination with other active ingredients useful for treatment of chronic pain, another embodiment of the invention relates to new pharmaceutical compositions comprising a therapeutically effective amount of flibanserin 1 as one active ingredient in combination with a therapeutically effective amount of at least one additional active ingredient 2 for the treatment of chronic pain and methods for the preparation thereof.

The compositions according to the invention may contain flibanserin 1 and the one or more additional active ingredient 2 in a single formulation or in separate formulations (multiple dosage form). If flibanserin 1 and the one or more additional one additional active ingredient 2 which is effective in the treatment of chronic pain, are present in separate formulations these separate formulations may be administered simultaneously or sequentially.

As an example such a multiple dosage form, e.g. a kit of parts may comprise
(a) a first containment containing a pharmaceutical composition comprising a therapeutically effective amount of flibanserin 1, optionally in form of the free base, the pharmacologically acceptable acid addition salts and/or optionally in form of the hydrates and/or solvates thereof and and optionally one or more pharmaceutically acceptable excipients, and
(b) a second containment containing a pharmaceutical composition comprising a therapeutically effective amount of at least one additional active ingredient 2 which is effective in the treatment of chronic pain, optionally in form of the pharmaceutically acceptable acid addition salts, in form of the hydrates and/or solvates and optionally in the form of the individual optical isomers, mixtures of the individual enantiomers or racemates thereof, and optionally one or more pharmaceutically acceptable excipients.

Accordingly, the present invention is directed to pharmaceutical compositions comprising a therapeutically effective amount of flibanserin 1 in combination with a therapeutically effective amount of one or more, preferably one active ingredient 2 selected from the group consisting of opioids, anticonvulsants, antidepressants, non-stereoidal antiflammatory drugs (“NSAIDs”) and anesthetics as all of such drugs can be used in combination with flibanserin for the treatment of chronic pain.

A preferred embodiment according to the invention is directed to pharmaceutical compositions comprising a therapeutically effective amount of flibanserin 1 and a therapeutically effective amount of one or more, preferably one opioid 2a, optionally in combination with a pharmaceutically acceptable excipient. Examples of suitable additional opioids include morphin, tramadol and buprenorphin, optionally in form of the pharmaceutically acceptable salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one anticonvulsant 2b, optionally in combination with a pharmaceutically acceptable excipient.

Examples of suitable additional anticonvulsants include gabapentin, carbamazepine, and phenytoin, optionally in form of the pharmaceutically acceptable salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one antidepressant 2c, optionally in combination with a pharmaceutically acceptable excipient.

Examples of suitable additional antidepressants include paroxetin, citalopram, and amitriptyline, most preferrably amitriptyline, optionally in form of the pharmaceutically acceptable salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one non-stereoidal antiflammatory drug 2d, optionally in combination with a pharmaceutically acceptable excipient.

Examples of suitable additional non-stereoidal antiflammatory drugs include meloxicam, diclofenac and ibuprofen, optionally in form of the pharmaceutically acceptable salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one local anesthetics 2e, optionally in combination with a pharmaceutically acceptable excipient.

Examples of suitable additional local anesthetics include lidocaine and mexiletine, optionally in form of the pharmaceutically acceptable salts, in form of the hydrates and/or solvates and optionally in the form of the individual optical isomers, mixtures of the individual enantiomers or racemates thereof.

Flibanserin 1 may be used in form of the free base, the pharmacologically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin 1 is used in form of the free base, it is preferably used in form of flibanserin polymorph A as disclosed in WO 03/014079.

The active ingredients 2 which are suitable to be combined with flibanserin within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds 2 carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds 2 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of the compounds 1 and 2. In general, such prodrugs will be functional derivatives of the compounds of this invention which are readily convertible in vivo into the required compound.

The term “therapeutically effective amount” shall mean that amount of a drug or pharmaceutical agent that will elicit the biological or medical response of a tissue, system, animal or human that is being sought by a researcher or clinician.

As used herein, the term “composition” is intended to encompass a product comprising the specified ingredients in the specified amounts, as well as any product which results, directly or indirectly, from combination of the specified ingredients in the specified amounts.

According to the present invention the component 1 may be administered as a monotherapy or together with component 2 as a combination therapy. If flibanserin 1 is administered in combination with component 2, 1 and 2 may be administered separately or together in one pharmaceutical composition. In addition, the administration of one element of the combination of the present invention may be prior to, concurrent to, or subsequent to the administration of the other element of the combination.

Flibanserin 1 or the elements of the combination of 1 and 2 may be administered by oral, parenteral (e.g., intramuscular, intraperitoneal, intravenous or subcutaneous injection, or implant), buccal, nasal, vaginal, rectal, sublingual, or topical (e.a. ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions, dosage forms, kit of parts for the administration of 1 or 1 and 2 of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions, dosage forms, kit of parts are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical formulations, compositions, dosage forms or kit of parts containing 1 and/or 2, separately or together, that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known to the art for the manufacture of pharmaceutical formulations and such compositions.

The excipients used may be for example, (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

In some cases, formulations for oral use may be in the form of hard gelatin or HPMC capsules wherein the active ingredients 1 and/or 2, separately or together, are mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Aqueous suspensions normally contain the active materials 1 and/or 2, separately or together, in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredients 1 and/or 2, separately or together, in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredient 1 and/or 2, separately or together in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical formulations, compositions, dosage forms or kit of parts of the invention may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical formulations, compositions, dosage forms or kit of parts containing 1 and/or 2, separately or together may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Preparations according to this invention containing 1 and/or 2, separately or together, for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstituted in sterile water, or some other sterile injectable medium immediately before use. The combination of this invention may also be administered in the form of suppositories for rectal administration. This composition can be prepared by mixing the drugs with a suitable non-irritating excipient which is solid at ordinary temperatures but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the formulations, compositions, dosage forms or kit of parts of this invention containing 1 and/or 2, separately or together may be formulated in liquid or semi-liquid preparations such as liniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the active ingredient 1 for the administration as a monotherapy or the active ingredients 1 or 2, for the administration as a combination therapy, be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respectively when the compounds are used singly.

Within the instant invention flibanserin 1 is preferably administered in such an amount that per single dosage between 0.01 to 400 mg of flibanserin 1 are applied. Preferred are ranges of between 0.1 to 300 mg, more preferred between 0.1 to 200 mg and particularly preferred 0.1 to 50 mg of flibanserin 1. Suitable dosage forms may contain for instance 0.01, 0.05, 0.1, 0.5, 1, 5, 10, 15, 20, 25, 30, 35, 40, 45, 50, 55, 60, 65, 70, 75, 80, 85, 90, 95, 100, 200, 300 or 400 mg of flibanserin 1. The aforementioned values are based on flibanserin 1 in form of the free base. If flibanserin 1 is applied in form of one of its acid addition salts, the corresponding values are readily calculable from the aforementioned values.

Within the instant invention the opioid 2a is preferably administered in such an amount that per day between 0.05 to 600 mg are applied. Preferred are ranges of between 0.1 to 300 mg, particular preferred 1 to 300 mg of the opioid 2a. In case of the preferred opioid 2a morphin particularly preferred doses per day are in the range of about 1 to 20 mg. In case of the preferred opioid 2a tramadol particularly preferred doses per day are in the range of about 1 to 600 mg. In case of the preferred opioid 2a buprenorphin particularly preferred doses per day are in the range of about 0.2 to 0.4 mg. Suitable dosage forms may contain for instance 0.1, 0.2, 0.3, 0.4, 0.5, 0.75, 1, 5, 10, 15, 20, 25, 30, 35, 40, 45, 50, 100, 200, 300, 400, 500 or 600 mg of the opioid 2a. Advantageously, the compounds 2a of the present invention may be administered in a single daily dose, or the total daily dosage may be administered in divided doses of two, three or four times daily.

Within the instant invention the anticonvulsant 2b is preferably administered in such an amount that per day between 1-2000 mg are applied. Preferred are ranges of between 10-1500 mg, particular preferred 50 to 600 mg of the anticonvulsant 2b. In case of the preferred anticonvulsant 2b gabapentin particularly preferred doses per day are in the range of about 50-600 mg. In case of the preferred anticonvulsant 2b carbamazepine, particularly preferred doses per day are in the range of about 600-1500 mg. In case of the preferred anticonvulsant 2b phenytoin particularly preferred doses per day are in the range of about 300-400 mg. Suitable dosage forms may contain for instance 1, 5, 10, 15, 20, 25, 30, 35, 40, 45, 50, 100, 200, 300, 400, 500 or 600 mg of the anticonvulsant 2b. Advantageously, the compounds 2b of the present invention may be administered in a single daily dose, or the total daily dosage may be administered in divided doses of two, three or four times daily.

Within the instant invention the antidepressant 2c is preferably administered in such an amount that per day between 1 to 200 mg are applied. Preferred are ranges of between 5 to 150 mg, particular preferred 10 to 100 mg of the antidepressant 2c. In case of the preferred antidepressant 2c amitriptyline particularly preferred doses per day are in the range of about 50 to 100 mg. In case of the preferred antidepressant 2c paroxetine particularly preferred doses per day are in the range of about 20 to 60 mg. In case of the preferred antidepressant 2c citalopram particularly preferred doses per day are in the range of about 10 to 40 mg. Suitable dosage forms may contain for instance 1, 5, 10, 15, 20, 25, 30, 35, 40, 45, 50, or 100 mg of the antidepressant 2c. Advantageously, the compounds 2c of the present invention may be administered in a single daily dose, or the total daily dosage may be administered in divided doses of two, three or four times daily.

Within the instant invention the non-stereoidal antiflammatory drugs 2d is preferably administered in such an amount that per day between 1 to 2000 mg are applied. Preferred are ranges of between 5 to 1500 mg, particular preferred 10 to 1200 mg of the non-stereoidal antiflammatory drugs 2d. In case of the preferred non-stereoidal antiflammatory drug 2d meloxicam particularly preferred doses per day are in the range of about 1-20 mg. In case of the preferred non-stereoidal antiflammatory drug 2d diclofenac particularly preferred doses per day are in the range of about 100-200 mg. In case of the preferred non-stereoidal antiflammatory drug 2d ibuprofen particularly preferred doses per day are in the range of about 600-1200 mg. Suitable dosage forms may contain for instance 1, 5, 10, 15, 20, 25, 30, 35, 40, 45, 50, 100, 200 and 500 mg of the non-stereoidal antiflammatory drugs 2d. Advantageously, the compounds 2d of the present invention may be administered in a single daily dose, or the total daily dosage may be administered in divided doses of two, three or four times daily.

Within the instant invention the local anesthetic 2e is preferably administered in such an amount that per day between 1 to 1500 mg are applied. Preferred are ranges of between 2 to 1000 mg, particular preferred 10 to 800 mg of the local anesthetics 2e. In case of the preferred local anesthetic 2e mexiletine preferred doses per day are in the range of about 100 to 800 mg, particularly in the range of about 200 to 700 mg. In case of the preferred local anesthetic 2e lidocaine preferred doses per day are in the range of about 2 ml of a 2% to 5% (w/v) solution. Suitable dosage forms may contain for instance 1, 5, 10, 15, 20, 25, 30, 35, 40, 45, 50, 100, 200, 500 and 1000 mg of the local anesthetic 2e. Advantageously, the compounds 2e of the present invention may be administered in a single daily dose, or the total daily dosage may be administered in divided doses of two, three or four times daily.

In an another embodiment, the invention relates to a method for the treatment of chronic pain comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one compound 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition as a combination therapy.

In another embodiment the invention is directed to a method for the treatment of chronic pain wherein the type of chronic pain is selected from the group consisting of neuropathic pain, diabetic neuropathy, post-herpetic neuralgia (PHN), carpal tunnel syndrome (CTS), HIV neuropathy, phantom limb pain, complex regional pain syndrome (CPRS), trigeminal neuralgia/trigeminus neuralgia/tic douloureux, surgical intervention (e.g. post-operative analgesics), diabetic vasculopathy, capillary resistance or diabetic symptoms associated with insulitis, pain associated with angina, pain associated with menstruation, pain associated with cancer, dental pain, headache, migraine, trigeminal neuralgia, temporomandibular joint syndrome, myofascial pain muscular injury, fibromyalgia syndrome, bone and joint pain (osteoarthritis), rheumatoid arthritis, rheumatoid arthritis and edema resulting from trauma associated with burns, sprains or fracture bone pain due to osteoarthritis, osteoporosis, bone metastases or unknown reasons, gout, fibrositis, myofascial pain, thoracic outlet syndromes, upper back pain or lower back pain (wherein the back pain results from systematic, regional, or primary spine disease (radiculopathy), pelvic pain, cardiac chest pain, non-cardiac chest pain, spinal cord injury (SCI)-associated pain, central post-stroke pain, cancer neuropathy, AIDS pain, sickle cell pain and geriatric pain, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one compound 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one compound 2, wherein 2 is selected from the group consisting of opioids, antidepressants, anticonvulsants, non-stereoidal antiflammatory drugs and local anesthetics.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one opioid 2a, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one opioid 2a selected from the group consisting of morphin, tramadol and buprenorphin, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one anticonvulsant 2b, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one anticonvulsant 2b selected from the group consisting of gabapentin, carbamazepine and phenytoin, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one antidepressant 2c, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one antidepressant 2c selected from the group consisting of amitriptyline, paroxetin and citalopram, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one non-steroidal antiinflammatory drug 2d, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one non-steroidal antiinflammatory drug 2d selected from the group consisting of meloxicam, diclofenac and ibuprofen, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one local anesthetic 2e, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another preferred embodiment of the invention is directed to a method for the treatment of any of the aforementioned disorders, comprising the administration of a therapeutically effective amount of 1, optionally in form of the free base, the pharmacologically acceptable acid addition salts and/or optionally in form of the hydrates and/or solvates thereof, in combination with a therapeutically effective amount of one or more, preferably one local anesthetic 2e selected from the group consisting of lidocaine and mexiletine, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another embodiment of the invention relates to the use of any of the aforementioned combinations of a therapeutically effective amount of flibanserin 1, optionally in form of the free base, the pharmacologically acceptable acid addition salts and/or optionally in form of the hydrates and/or solvates thereof, and a therapeutically effective amount of a compound 2, optionally in form of their pharmaceutically acceptable acid addition salts for the preparation of a medicament for the treatment of any of the aforementioned disorders. Preferred compounds 2 are selected from the group consisting of opioids 2a, anticonvulsants 2b, antidepressants 2c, non-stereoidal antiflammatory drugs 2d, and local anesthetics 2e. Preferred opioids are morphin, tramadol and buprenorphin. Preferred anticonvulsants are gabapentin, carbamazepine and phenytoin. Preferred antidepressants are paroxetin, citalopram and amitriptyline, most preferrably amitriptyline, optionally in form of the pharmaceutically acceptable salts. Preferred non-stereoidal antiflammatory drugs are meloxicam, diclofenac and ibuprofen. Preferred local anesthetics are lidocaine and mexiletine.

The invention further relates to the use of a therapeutically effective amount of flibanserin 1, optionally in form of the free base, the pharmacologically acceptable acid addition salts and/or optionally in form of the hydrates and/or solvates thereof for the manufacture of a medicament for the treatment of any of the aforementioned disorders, in combination with a therapeutically effective amount of a compound 2 which is effective in the treatment of chronic pain, in a patient. Preferred compounds 2 are selected from the group consisting of opioids 2a, anticonvulsants 2b, antidepressants 2c, non-stereoidal antiflammatory drugs 2d, and local anesthetics 2e. Preferred opioids are morphin, tramadol and buprenorphin. Preferred anticonvulsants are gabapentin, carbamazepine and phenytoin. Preferred antidepressants are paroxetin, citalopram and amitriptyline, most preferrably amitriptyline, optionally in form of the pharmaceutically acceptable salts. Preferred non-stereoidal antiflammatory drugs are meloxicam, diclofenac and ibuprofen. Preferred local anesthetics are lidocaine and mexiletine.

The beneficial effects of the invention can be observed regardless of whether the disturbance existed lifelong or was acquired, and independent of etiologic origin (organic—both, physically and drug induced-, psychogen, a combination of organic—both, physically and drug induced-, and psychogen, or unknown).

The following examples demonstrate possible pharmaceutical compositions comprising flibanserin for monotherapy or in combination with one of the aforementioned combination partners 2.

### EXAMPLE NO 1

### Combination 1 with 2a

| | |
|---|---|
| Constituents | mg/tablet |

Core

| | |
|---|---|
| Flibanserin (free base) | 50.000 |
| morphin | 20.000 |
| Anhydrous dibasic calcium phosphate | 100.000 |
| Microcrystalline cellulose | 203.090 |
| HPMC (Methocel E5) | 6.615 |
| Croscarmellose sodium | 8.820 |
| Magnesium stearate | 2.250 |

Coating

| | |
|---|---|
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 399.775 |

### EXAMPLE NO 2

### Combination 1 with 2b

| | |
|---|---|
| Constituents | mg/tablet |

Core

| | |
|---|---|
| Flibanserin (free base) | 50.000 |
| gabapentin | 50.000 |
| Lactose monohydrate | 133.750 |
| Microcrystalline cellulose | 40.000 |
| Hydroxypropylcellulose | 2.500 |
| Corn starch | 12.500 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide yellow | 0.043 |
| Total Film coated tablet | 295.000 |

### EXAMPLE NO 3

### Combination 1 with 2c

| | |
|---|---|
| Constituents | mg/tablet |

Core

| | |
|---|---|
| Flibanserin (free base) | 50.000 |
| Amitriptyline | 50.000 |
| Lactose monohydrate | 143.490 |
| Microcrystalline cellulose | 47.810 |
| HPMC (e.g. Pharmacoat 606) | 2.500 |
| Carboxymethylcellulose sodium | 5.000 |
| Mannitol | 60.000 |
| Corn starch | 36.500 |
| Povidone | 1.000 |
| Colloidal silicon dioxide | 1.000 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated bilayer tablet | 406.000 |

### EXAMPLE NO 4

### Combination of 1 with 2d

| | |
|---|---|
| Constituents | mg/tablet |

Final Mixture

| | |
|---|---|
| Flibanserin (free base) | 50.000 |
| Diclofenac | 100.000 |
| Lactose monohydrate | 200.000 |
| Pregelatinized starch | 108.000 |
| Magnesium stearate | 2.000 |

Capsule

| | |
|---|---|
| Final Mixture | 460.000 |
| Capsule (size 1) | 82.000 |
| Total weight of Capsule | 542.000 |

The following examples show preferred pharmaceutical compositions of flibanserin, if the combinations according to the invention are administered in separate dosage units.

### EXAMPLE NO 5

### Composition

| | |
|---|---|
| Constituents | mg/tablet |

Core

| | |
|---|---|
| Flibanserin (free base) | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

### EXAMPLE NO 6

### Composition

| | |
|---|---|
| Constituents | mg/tablet |

Core

| | |
|---|---|
| Flibanserin (free base) | 50.000 |
| Lactose monohydyrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

### EXAMPLE NO 7

### Composition

| | |
|---|---|
| Constituents | mg/tablet |

Core

| | |
|---|---|
| Flibanserin (free base) | 100.000 |
| Lactose monohydyrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

### EXAMPLE NO 8

### Composition

| | |
|---|---|
| Constituents | mg/tablet |

Core

| | |
|---|---|
| Flibanserin (free base) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

### EXAMPLE NO 9

### Composition

| | |
|---|---|
| Constituents | mg/tablet |

Core

| | |
|---|---|
| Flibanserin (free base) | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

### EXAMPLE NO 10

### Composition

| | |
|---|---|
| Constituents | mg/tablet |

Core

| | |
|---|---|
| Flibanserin (free base) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

What is claimed is:

1) A pharmaceutical composition comprising flibanserin 1, in form of the free base, a pharmacologically acceptable acid addition salt or in form of a hydrate or solvate thereof, or a combination thereof, as one active ingredient in combination with at least one second active ingredient 2, which second active ingredient is effective in the treatment of chronic pain.

2) The pharmaceutical composition according to claim 1, wherein the active ingredient 2 is selected from the group consisting of opioids 2a, anticonvulsants 2b, antidepressants 2c, non-stereoidal antiflammatory drugs 2d, local anesthetics 2e, and combinations thereof.

3) The pharmaceutical composition according to claim 2, wherein the opioid 2a is selected from the group consisting of morphin, tramadol and buprenorphin, in form of a pharmaceutically acceptable acid addition salt, in form of a hydrate or solvate or in the form of an individual optical isomer, a mixture of the individual enantiomers, or a racemates thereof, or a combination thereof.

4) The pharmaceutical compositions according to claim 2, comprising a therapeutically effective amount of flibanserin 1 and a therapeutically effective amount of one or more anticonvulsant 2b.

5) The pharmaceutical compositions according to claim 4, wherein the anticonvulsant 2b is selected from the group consisting of gabapentin, carbamazepine, phenytoin, or combinations thereof.

6) The pharmaceutical compositions according to claim 2, comprising a therapeutically effective amount of flibanserin 1 and a therapeutically effective amount of one or more antidepressant 2c.

7) The pharmaceutical compositions according to claim 6, wherein the antidepressant 2c is selected from the group consisting of paroxetine, citalopram, amitriptyline, or combinations thereof.

8) The pharmaceutical composition according to claim 2, comprising a therapeutically effective amount of flibanserin 1 and a therapeutically effective amount of one or more non-stereoidal antiflammatory drug 2d.

9) The pharmaceutical compositions according to claim 8, wherein the non-stereoidal antiflammatory drug 2d is selected from the group consisting of meloxicam, diclofenac, ibuprofen, or combinations thereof.

10) The pharmaceutical composition according to claim 2, comprising a therapeutically effective amount of flibanserin 1 and a therapeutically effective amount of one or more local anesthetic 2e.

11) The pharmaceutical composition according to claim 10, wherein the local anesthetic 2e is selected from the group consisting of lidocaine, mexiletine, or combinations thereof.

12) A method for the treatment of chronic pain comprising the administration of a therapeutically effective amount of flibanserin 1, in form of the free base, a pharmacologically acceptable acid addition salt or in form of the hydrate or solvate thereof, or a combination thereof.

13) A method for the treatment of chronic pain comprising the administration of a therapeutically effective amount of flibanserin 1, in form of the free base, a pharmacologically acceptable acid addition salt or in form of a hydrate or a solvate thereof, in combination with a therapeutically effective amount of another active ingredient 2, which another active ingredient is effective in the treatment of chronic pain.

14) The method according to claim 13, wherein 2 is selected from the group consisting of opioids 2a, anticonvulsants 2b, antidepressants 2c, non-stereoidal antiflammatory drugs 2d, local anesthetics 2e, and combinations thereof.
